(19) United States
(12) Patent Application Publication
(10) Pub. No.: US 2005/0197321 A1
(43) Pub. Date: Sep. 8, 2005

(54) Treatment of sepsis

(76) Inventors: Robert S. Riggs, Granbury, TX (US); George Moore, Granbury, TX (US)

Correspondence Address: CARSTENS YEE & CAHOON, LLP, P O BOX 802334, DALLAS, TX, 75380, US

(21) Appl. No.: 11/007,476

(22) Filed: Dec. 8, 2004

Related U.S. Application Data
(60) Provisional application No. 60/550,087, filed on Mar. 4, 2004.

(51) Int. Cl. A61K 31/198; A61K 31/66

(52) U.S. Cl. 514/114; 514/565; 424/451

(57) ABSTRACT

The present invention provides a treatment for sepsis comprising an intravenous solution or pill that contains creatine phosphate and is devoid of carbohydrates. The intravenous solution can be used as a common carrier for antibiotics used to treat the septic condition. Because bacteria cannot utilize creatine phosphate to generate ATP, the creatine phosphate in the solution or pill provides an energy source for the patient but not the bacteria. The present invention avoids the problem of standard glucose solutions, which provide a ready nutritional source for the infecting bacteria.

### CROSS-REFERENCE TO RELATED APPLICATION

This application claims the benefit of and priority to a U.S. Provisional Patent Application No. 60/550,087 filed Mar. 04, 2004, the technical disclosure of which is hereby incorporated herein by reference.

### TECHNICAL FIELD

The present invention relates generally to the treatment of sepsis in medical patients and more specifically to the use of creatine phosphate or salts thereof to provide nutrition to the patient without stimulating the growth of microorganisms.

### BACKGROUND OF THE INVENTION

Sepsis, also known as Systemic Inflammatory Response Syndrome (SIRS), is a severe illness caused by infection of the bloodstream by toxic bacteria or other microorganisms. Sepsis may constitute a life threatening condition, especially in people with a weakened immune system or other medical illness. The effects of sepsis can range from systemic inflammation to organ dysfunction to multiple organ failure, and ultimately death for many patients.

The conventional treatment of sepsis involves an intravenous (IV) regimen of broad spectrum antibiotics used in conjunction with a common carrier fluid such as 5% dextrose in water (D5W). Carbohydrates, especially glucose (dextrose), are the preferential food source of cells. As such, dextrose has been incorporated into a number of standard IV bag solutions, providing nourishment and energy to support cellular metabolism. Unfortunately, the carbohydrates provided by these various IV solutions can also be utilized by bacteria and other microorganisms. Thus the inclusion of carbohydrates, especially glucose, in an IV solution provides nutrition to the very microbes the therapy is attempting to eliminate, thus partially negating the benefits of the therapy.

Therefore, it would be desirable to have a method for treating septic conditions and providing energy substrates for cellular metabolism, without using carbohydrates that can also be used by the infectious microbes.

### SUMMARY OF THE INVENTION

The present invention provides a treatment for sepsis comprising an intravenous solution or pill that contains creatine phosphate or salts thereof and is devoid of carbohydrates. The intravenous solution can be used as a common carrier for antibiotics used to treat the septic condition. Because microorganisms do not contain the enzyme necessary to utilize creatine phosphate's high energy phosphate group to phosphorylate ADP back to ATP, the creatine phosphate provides an energy source for the patient but not the microbes. The present invention avoids the problem of standard glucose solutions, which readily stimulate the growth of bacteria and other infectious microorganisms.

### BRIEF DESCRIPTION OF THE DRAWINGS

The novel features believed characteristic of the invention are set forth in the appended claims. The invention itself, however, as well as a preferred mode of use, further objects and advantages thereof, will best be understood by reference to the following detailed description of an illustrative embodiment when read in conjunction with the accompanying drawings, wherein:

FIG. 1 shows a typical intravenous (IV) administration bag which may be used with the present invention.

### DETAILED DESCRIPTION OF THE DRAWINGS

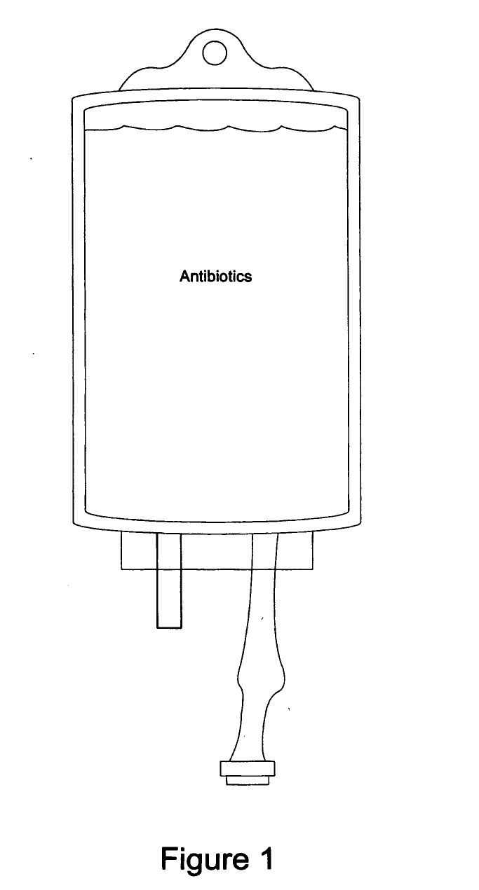

FIG. 1 shows a typical intravenous (IV) administration bag which may be used with the present invention. The standard course of treatment for patients with sepsis comprises IV administration of broad spectrum antibiotics, usually-in conjunction with a common carrier fluid such as 5% dextrose in water (D5W). D5W is an isotonic solution and may also contain electrolytes comprised of sodium chloride and potassium chloride as well as salts of calcium or magnesium. The concentration of sugars and electrolytes may be adjusted to achieve the desired level of osmolarity.

For the purposes of energy generation, carbohydrates are the preferential food sources of virtually all biological cells. In a solution of various nutrient substrates (i.e. carbohydrates, lipids, proteins, and nucleic acids), cells will tend to process essentially all of the carbohydrates available before resorting to the oxidation of lipids followed by proteins and nucleic acids. It should be noted that for purposes other than energy production (e.g., protein or steroid synthesis), various substrates may be metabolize more or less simultaneously.

Glucose is metabolized catabolically via glycolysis and the Kreb's cycle to generate chemical energy stored in the form of adenosine triphosphate (ATP). ATP is the equivalent of a biological battery with a Gibb's free energy of hydrolysis of −7.3 kcal/mol. This energy can then be made available to drive various biochemical reactions which would not otherwise occur spontaneously, at that temperature. Thus, ATP serves as the universal currency of energy storage at the cellular level.

The amount of energy available is defined as the change in enthalpy, minus the absolute temperature times the change in entropy.
ΔG=ΔH−TΔS
where:
G=Gibb's free energy (kcal/mol)
H=enthalpy (kcal/mol)
T=absolute temperature, Kelvin
S=entropy (kcal/deg/mol)

If ΔG is negative, the reaction will proceed spontaneously as written, from left to right. It is a matter of convention that a negative energy value refers to an exothermic reaction wherein energy is being release. A positive ΔG indicates that additional external energy is required to drive the reaction from left to right; the same reaction could proceed spontaneously from right to left.

Energy is released from the ATP molecule by cleaving off one of the phosphate molecules to produce adenosine diphosphate (ADP):
ATP⇄ADP+Pᵢ+Energy

This reaction in catalyzed by ATPase enzymes. ATP is continuously consumed and regenerated at a very rapid rate, since the total ATP/ADP pool is extremely small and can maintain active tissue for only a few seconds.

Members of the phylum chordata (vertebrates) have developed an alternate method of storing high energy phosphate bonds in the form of creatine phosphate (CP), also referred to as phosphocreatine (PC). Creatine phosphate has a free energy of hydrolysis of −10.3 kcal/mol. Therefore, CP releases 3 kcal/mol more energy than ATP under similar conditions.

CP is found most abundantly in muscle and nervous system tissues to provide a mechanism for the cell to rephosphorylate ADP back to ATP without having to wait for further substrate metabolism to provide energy.
CP+ADP⇄Creatine+ATP

Under physiologic conditions, CP permits ATP concentrations to be maintained when ATP is rapidly utilized as an energy source. This method of rephosphorylating ADP requires the presence of the enzyme creatine phosphorkinase, usually referred to simply as creatine kinase (CK), an enzyme not present in microbial cells.

CK may occur in any of three different isoenzyme forms, each existing as a dimer consisting of two strands of polypeptide. Each strand is further designated as being type “m” or type “b”. The CK dimer may be composed of two strands of the “m” types (CKmm), two strands of the “b” type (CKbb), or one strand of each (CKmb). CKmm occurs in skeletal muscle and plasma, CKbb occurs in brain, prostate thyroid, gut and lung tissues, and CKmb occurs in cardiac muscle.

Unfortunately for septic patients, the bacteria causing the septic reaction can also metabolize the glucose in standard IV solutions to produce ATP. However, unlike vertebrate cells, microbes do not contain the creatine kinase necessary to phosphorylate ADP back to ATP. The present invention takes advantage of this deficiency in microbial metabolism by substituting creatine phosphate (and/or salts thereof, e.g., calcium or sodium salts) for glucose and other carbohydrates in IV solutions used in the treatment of sepsis. The CP in the IV solution provides a readily usable energy source for the cells of the patient but is not similarly usable by the microbes, allowing the therapy to be more effective.

Typical dosages for IV administration of creatine phosphate or salts thereof range from 1 gram/liter to 100 gram/liter, with possible utility being found from 1 mg/liter to the saturation limit which may approach 1 kg/liter.

In addition to the IV administration described above, creatine phosphate or its salts may be administered orally in the form of an enteric coated capsule or solid tablet. Because creatine phosphate is cleaved by gastric enzymes, the enteric coating allows the tablet or capsule to pass through the stomach and into the intestine where a more suitable chemical environment will permit the CP to be absorbed. Enteric coating is a standard method used in the art to protect chemicals from premature breakdown in the stomach, as well as to avoid irritation to the stomach from particular compounds (e.g., aspirin). Oral preparations of CP may also utilize time release methods, which are well known in the art.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

1. A treatment for sepsis, comprising:
an intravenous solution containing creatine phosphate and/or salts thereof, wherein the intravenous solution is devoid of carbohydrates.

2. The treatment according to claim 1, wherein the intravenous solution further contains antibiotics.

3. The treatment according to claim 1, wherein the intravenous solution further contains electrolytes.

4. The treatment according to claim 1, wherein concentration of creatine phosphate is between 1 gram/liter and 100 gram/liter, inclusive.

5. The treatment according to claim 1, wherein the concentration of creatine phosphate is between 1 mg/liter and 1 kg/liter, inclusive.

6. A treatment for sepsis, comprising:
an enteric coated pill containing creatine phosphate and/or salt thereof, wherein the tablet is devoid of carbohydrates.

7. The treatment according to claim 6, wherein the pill is a solid tablet.

8. The treatment according to claim 6, wherein the pill is a capsule.

9. The treatment according to claim 6, wherein the creatine phosphate is time released.
